(19) United States
(12) Patent Application Publication
(10) Pub. No.: US 2024/0009135 A1
(43) Pub. Date: Jan. 11, 2024

(54) DENSIFIED CO-PRECIPITATED MATERIALS ISOLATED BY THIN FILM EVAPORATION

(71) Applicant: Merck Sharp & Dohme LLC, Rahway, NJ (US)

(72) Inventors: Derek S. Frank, Brooklyn, NY (US); Haichen Nie, Exton, PA (US); Luke Ryan Schenck, Yardley, PA (US); Jasmine M. Rowe, Robbinsville, NJ (US)

(73) Assignee: Merck Sharp & Dohme LLC, Rahway, NJ (US)

(21) Appl. No.: 18/348,787

(22) Filed: Jul. 7, 2023

Related U.S. Application Data
(60) Provisional application No. 63/359,548, filed on Jul. 8, 2022.

(51) Int. Cl. A61K 9/20; A61K 31/513

(52) U.S. Cl. CPC A61K 9/2095; A61K 9/2054; A61K 31/513

(57) ABSTRACT

The invention encompasses a process for densifying co-precipitated material comprised of an active pharmaceutical ingredient and at least one stabilizing excipient using thin film evaporation, and the densified co-precipitates made thereby. Bulk density and flowability of co-precipitates can be increased by processing the co-precipitated material using thin film evaporation.

### CROSS-REFERENCE TO RELATED APPLICATIONS

This application claims the benefit of priority to U.S. Provisional Patent Application No. 63/359,548, filed Jul. 8, 2022.

### BACKGROUND OF THE INVENTION

Co-processing of an active pharmaceutical ingredient (API) with a polymer is an emerging route to enhance bioavailability of active pharmaceutical ingredients, including those that are poorly soluble, and improve material attributes. Co-processing can be achieved by simultaneous precipitation of an active pharmaceutical ingredient and a stabilizing excipient, also known as co-precipitation, to generate a composite material containing microcrystalline API, nanocrystalline API, or amorphous API dispersed in an excipient matrix.

Co-precipitation is a solvent-based process where a solvent stream containing dissolved API and excipient is rapidly introduced into anti-solvent in a high shear field (e.g., see Dong, Z. et al., Evaluation of solid-state properties of solid dispersions prepared by hot-melt extrusion and solvent co-precipitation, International Journal of Pharmaceutics 2008, 355 (1-2), 141-149). Although high shear precipitation is an optimal method to ensure intimate contact between precipitated drug and stabilizing excipient, it can also result in material morphologies which show non-optimal bulk powder properties. Co-precipitated materials can be isolated from mother liquors by filtration, evaporative isolation, or any other drying technique. Though filtration is often used to remove remaining mother liquors from the precipitate, filtered co-precipitated materials can suffer from challenging material attributes such as low bulk density and poor flowability and require additional downstream processing.

Previous work has investigated thin film evaporation as an isolation route to remove solvents and control particle properties of multi-component pharmaceutical materials containing micronized, nanosized, or amorphous drug substance (see, e.g., US Patent Publication No. 2020/0261365 A1 and Schenck, L. Building a better particle: Leveraging physicochemical understanding of amorphous solid dispersions and a hierarchical particle approach for improved delivery at high drug loadings. International Journal of Pharmaceutics 2019, 559, 147-155.). Additionally, previous work has leveraged thermal annealing as a strategy to improve bulk density of amorphous materials (see, e.g., Frank, D., Optimizing Solvent Selection and Processing Conditions to Generate High Bulk-Density, Co-Precipitated Amorphous Dispersions of Posaconazole, Pharmaceutics 2021, 13(12), 2017). Improvements to the processing of co-precipitated materials from solvent, both to enable continuous manufacturing as well as to optimize powder attributes of the co-precipitated material, are desired in order to simplify manufacturing trains, improve robustness and decrease cost. Co-precipitated amorphous dispersions are a class of co-precipitated materials, and such improvements are likewise desired for co-precipitated amorphous dispersions.

### SUMMARY OF THE INVENTION

The present disclosure is directed to a novel process for densifying a co-precipitated material, including a co-precipitated amorphous dispersion (cPAD), wherein the co-precipitated material is comprised of an active pharmaceutical ingredient (API) and at least one stabilizing excipient. Densification of the co-precipitated material can be achieved during isolation of the co-precipitated material from solvent by annealing it above its wetted glass transition (T_{g}) temperature.

Densification and other desirable properties of the co-precipitated material can be achieved by the process of using thin film evaporation to dry and anneal the co-precipitated material above its wetted glass transition temperature. The present disclosure is also directed to a pharmaceutical composition comprised of the densified co-precipitated material generated by the disclosed thin film evaporation process.

### BRIEF DESCRIPTION OF THE FIGURES

FIG. 1a shows a Powder X-Ray Diffraction (PXRD) of Compound A/HPMCAS co-precipitated amorphous dispersion (cPAD) (30% DL) isolated by filtration and drying. Compound A is ulonivirine.

FIG. 1B shows a Modulated Differential Scanning calorimetry (MDSC) trace for Compound A/HPMCAS co-precipitated amorphous dispersions showing a glass transition temperature (T_{g}) of 99° C.

FIG. 2a shows a PXRD of Compound A/HPMCAS co-precipitated amorphous dispersion (30% DL) isolated by thin film evaporation (TFE).

FIG. 2b shows an MDSC trace for thin film evaporated Compound A/HPMCAS co-precipitated amorphous dispersions showing a glass transition temperature of 99° C.

FIG. 3a is a photograph of two 4 mL scintillation vials of cPAD Compound A/HPMCAS that compares the bulk density of filtered and dried cPAD and TFE-processed (or TFE-isolated) cPAD.

FIG. 3b shows a scanning electron microscope (SEM) image of the filtered and dried Compound A/HPMCAS cPAD.

FIG. 3c shows a scanning electron microscope image of cPAD/TFE powder.

FIG. 4 shows a dot plot that compares the compaction profiles of formulated Compound A/HPMCAS cPAD/TFE and spray-dried intermediate (SDI) blend.

### DETAILED DESCRIPTION OF THE INVENTION

Provided herein is a process for making drug materials with high bulk density (≥0.3 g/cc), high flowability, and sufficient mechanical strength to enable direct compression of high drug loaded tablets. More specifically, these drug materials are generated by co-precipitation of an active pharmaceutical ingredient (API) with one or more stabilizing excipient(s) which are then processed by thin film evaporation (TFE).

In some embodiments, the API is ulonivirine. Ulonivirine is also known as 3-chloro-5-((6-oxo-1-((6-oxo-5-(trifluoromethyl)-1,6-dihydropyridazin-3-yl)methyl)-4-(trifluoromethyl)-1,6-dihydropyrimidin-5-yl)oxy)benzonitrile. See International Patent Publication No. WO 2014/058747, which is incorporated by reference herein.

It is known that thin film evaporation has been used to remove solvent from pharmaceutical materials. The present disclosure is based, at least in part, on the discovery that the thin film evaporation process can be used to generate co-precipitated material (including co-precipitated amorphous dispersions) having improved material properties that have not been achievable using previously known processes. Herein is provided an improved method for processing a co-precipitated material using thin film evaporation to heat and remove solvent from a co-precipitated material to achieve densification, i.e., improved bulk density, of the co-precipitated material. This process additionally provides increased particle size and improved flowability of the co-precipitated material.

During processing by thin film evaporation, the co-precipitated material is annealed above its glass transition temperature (T_{g}) and undergoes densification. Shear forces in the thin film evaporator lead to an increase in particle size of the co-precipitated material and an improvement in its flowability. This annealing process can be achieved with (a) the removal of solvent, (b) increased temperature, or (c) removal of solvent and increased temperature. Processing co-precipitated materials above their T_{g} allows for densification which can improve their bulk density and powder flow properties to enable direct compression into tablets with improved drug loading.

Additionally, the shear environment in the thin film evaporator gives rise to a preferred granular morphology for the co-precipitated material. Co-precipitated materials processed using this thin film evaporation manufacturing pathway are amenable to direct compression at high drug loading in tablet dosage forms. The thin film evaporation process described herein greatly reduces or eliminates the need for bulking agents to improve bulk powder properties of co-precipitated materials. Without the need for bulking agents, flow aids, and mechanical strengtheners in a drug formulation, tablets containing the densified co-precipitated material have fewer excipients and are smaller in size, thus more likely to improve patient compliance to follow a dosing regimen for a prescribed medicine. Additionally, drying of pharmaceutical materials by thin film evaporation can reduce solvent levels to acceptable values. Densification of co-precipitated materials by thin film evaporation is of value for its ability to continuously produce co-precipitated materials that result in reduced oral tablet unit dosage size as well as improved manufacturability of pharmaceutical powders.

Common pharmaceutical unit operations such as roller compaction are used to improve flow properties of pharmaceutical powders. The approach provided herein results in improved product properties during drying of the co-precipitated material and thus removes any need for additional manufacturing steps from a process train to achieve material properties amenable to tablet compression. The impact of the glass transition on mechanical properties is a well-researched area in polymer chemistry and polymer physics. We have discovered a new way to apply this phenomenon to continuously densify pharmaceutical materials during isolation by processing via thin film evaporation. Such a unit operation can be enabled as part of a continuous manufacturing train to densify large quantities of co-precipitated material.

Herein is described an integrated process to produce a co-precipitated material by (1) introducing a solvent stream containing dissolved API and one or more stabilizing excipient(s) into anti-solvent to form a co-precipitated material, and (2) annealing the co-precipitated material by thin film evaporation above its wetted glass transition temperature. In an embodiment thereof, the API and the stabilizing excipient(s) are precipitated in a turbulent shear field. In another embodiment, rapid mixing to precipitate the material is achieved through the use of an in-line rotor-stator precipitation device at tip speeds greater than 1 m/s.

The stabilizing excipient(s) in the co-precipitated material is a non-active component included to control material attributes of the API such as crystallite size, crystal form, and chemical stability, or in the case of amorphous co-precipitated materials (e.g., co-precipitated amorphous dispersions) to prevent crystallization of API from the amorphous state during processing and storage.

In an embodiment, the co-precipitated material may comprise an API and one, two, or three stabilizing excipient(s). In some embodiments, the co-precipitated material comprises an API and one stabilizing excipient. As such, some embodiments of the disclosed process comprise introducing a solvent stream containing dissolved API and one, two, or three stabilizing excipient(s) into anti-solvent to form a co-precipitated material. Examples of stabilizing excipients include, but are not limited to, water-soluble polymers, water-insoluble polymers, polysaccharide or polysaccharide derivatives, generally recognized as safe (GRAS) molecules, or any other suitable component selected by those skilled in the art. In an embodiment thereof, the one or more stabilizing excipient(s) is one or more stabilizing polymer; or one, two or three stabilizing polymer(s); or one stabilizing polymer.

The disclosed co-precipitation process can be performed with any stabilizing polymer and any solvent/anti-solvent combination suitable for use with the chosen API. Examples of such polymers include, but are not limited to, hydroxypropyl methylcellulose acetate succinate (including each of HPMCAS-L, HPMCAS-M and HPMCAS-H), hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone, and polyvinylpyrrolidinone-polyvinylacetate copolymers.

In another embodiment, the process herein generates a co-precipitated material which is a co-precipitated amorphous dispersion (cPAD), wherein the API is dispersed in the stabilized excipient in its amorphous phase. In other embodiments, a nanocrystalline material, (e.g., nanocrystalline dispersion, nanocrystalline solid dispersion), or microcrystalline material, (e.g., microcrystalline dispersion, microcrystalline solid dispersion) embedded in a stabilizing excipient, (e.g., a polymer), can be processed by the same process described herein to achieve a similar densification and improvement in morphology. Appropriate solvent/anti-solvent combinations for co-precipitation can be readily selected by those skilled in the art. Some examples of solvent/anti-solvent combinations include, but are not limited to, acetone/water, methanol/0.001 N HCl, ethanol/0.1 vol % aqueous ammonia, tetrahydrofuran/n-heptane, and 2-butanone/methyl tert-butyl ether. In the example provided below, acetone is used as the solvent and acidified water (0.001 N HCl) is used as the anti-solvent.

As illustrated below, the densified co-precipitated material processed by thin film evaporation may not require roller compaction to achieve adequate flow properties for tableting. Additionally, formulations including these densified amorphous dispersions may not require mechanical strength modifiers in the formulation in order to form a tablet that can withstand packaging and shipping. The large particle size material achieved after processing with thin film evaporation has favorable flow properties and is highly amenable to downstream formulation. As a result, the final dosage unit can require fewer excipients and may be of a lower image size than tablets containing equivalent amorphous dispersions generated by other means. The properties of the densified co-precipitated material processed by thin film evaporation are amenable to direct compression in a manufacturing process. Direct compression is a preferred manufacturing strategy due to the resulting reduced need for additional excipients. Additionally, direct compression eliminates the need for roller compaction or other granulation approaches to enable tablet formation. This reduces cost and complexity of formulation for drug products.

In some aspects, provided herein are pharmaceutical compositions comprising the disclosed densified co-precipitated amorphous material, wherein the material comprises an API. In some embodiments are pharmaceutical compositions comprising a co-precipitated amorphous dispersion wherein the API is stabilized in its amorphous phase by at least one stabilizing excipient, and the cPAD has been dried above its wetted glass transition temperature by thin film evaporation. In a further embodiment thereof, the cPAD in the pharmaceutical composition is comprised of an API and one, two or three stabilizing excipient(s). In another embodiment thereof, the one, two or three stabilizing excipient(s) are stabilizing polymer(s).

In some embodiments, any of the disclosed pharmaceutical compositions may exhibit greater (or higher) bulk density relative to a corresponding pharmaceutical composition that has not been annealed above its wetted glass transition temperature by thin film evaporation.

The disclosed compositions may be formulated into compressed tablets. As such, further provided herein are compressed tablets comprising any of the disclosed co-precipitated amorphous materials. In some embodiments, compressed tablets comprising a pharmaceutical composition comprising an API dispersed in one or more stabilized excipients in accordance with the disclosure. In some aspects, the API is Compound A. Further provided herein are methods of formulating any of the disclosed pharmaceutical compositions into compressed tablets.

The process described herein can be applied to any co-precipitated material amenable to processing by thin film evaporation. The co-precipitation process can be performed in-line with processing on the thin film evaporator. The co-precipitation step is performed by dissolving the API and one or more stabilizing excipients in solvent and precipitating into anti-solvent in a shear field to produce the co-precipitated material. Thin film evaporation can be performed on co-precipitated material isolated from a batch crystallization vessel by controlled solvent addition. Additionally, co-precipitated material can be resuspended in a processing solvent and isolated by thin film evaporation to afford improved product properties.

### Terminology as Used Herein

Bulk density is the ratio of the mass of a bulk solid to its volume which determines the space occupied by a given amount of material.

Flow properties, also referred to as powder flow or flowability, is defined as the relative movement of a bulk of particles among neighboring particles or along the container wall surface. In other words, these terms refer to the ability of a powder to flow in a desired manner in a specific piece of equipment.

The term “co-precipitated material” refers to a material produced by co-precipitation of an API and one or more stabilizing excipients. The term “co-precipitated material” refers to co-precipitated amorphous dispersions and co-precipitates that are not amorphous dispersions.

The term “cPAD” refers to a co-precipitated amorphous dispersion. The term “FTE” refers to thin film evaporation. The term “cPAD/TFE” refers to cPAD processed by thin film evaporation.

As used herein, the term “co-precipitate” refers to a “co-precipitated material,” and in the case of an amorphous co-precipitate the term “co-precipitate” may also be used to refer to a co-precipitated amorphous dispersion (cPAD).

Densifying, densification and similar terms in the same context refer to a method for increasing the density of a material, resulting in the material having greater density than its original density, e.g., a densified co-precipitated amorphous material, or a densified co-precipitated amorphous dispersion. The term “density” encompasses each of bulk density and tapped density.

The term “amorphous” refers to a material lacking crystallinity in the solid-state, as determined by an analytical technique such as powder X-ray diffractometry, nuclear magnetic resonance spectroscopy, or vibrational spectroscopy. The acceptable bounds on limits of detection for an amorphous material can be defined as they relate to pharmacokinetic performance of a given pharmaceutical.

The term “material attributes” (also referred to herein as “material properties”) of a co-precipitated material refers to product properties of a co-precipitated material including, but not limited to, bulk density, dissolution rate, solubility, flowability, compressive strength and particle size, which have influence on the ability to manufacture oral dosage forms and dictate pharmacokinetic behavior of the pharmaceutical in a subject of interest.

Annealing (or anneal) is the process of heating a material (in this case a co-precipitated material, e.g., a co-precipitated amorphous dispersion), and allowing it to cool slowly, in order to remove internal stresses and toughen it.

The term “high shear” refers to a mixing environment that results in precipitation of a co-precipitate with well-defined properties as they relate to material attributes of the bulk powder.

The terms spray dried intermediate and spray dried dispersion are used interchangeably herein and generally mean an amorphous dispersion of an API in a stabilizing excipient such as a polymer matrix.

The term “image size,” “unit dosage size,” or “tablet size,” or some combination of those terms, refers to the mass of a formulated dosage unit required to administer a given quantity of pharmaceutical.

Additional abbreviations and acronyms used herein are defined as follows: w/w is weight for weight; wt. % is weight percent; ft is foot; 1/hr is liter(s) per hour; m/s is meters per second; g/cc is gram per cubic centimeter; DL is drug load; “i.e.” is that is; and “e.g.” is for example. Active pharmaceutical ingredient or “API” is used interchangeably with “drug.”

In instances where a word encompasses both a singular and plural meaning, the word may appear with a terminal “(s)”, e.g., “one, two or three stabilizing excipient(s).”

With respect to Table 1, D10 is the portion of particles with diameters smaller than this value is 10%; D50 is the portions of particles with diameters smaller and larger than this value are 50%; and D90 is the portion of particles with diameters below this value is 90%.

The following is an Example of the process described herein.

### Example 1: Co-Precipitated Amorphous Dispersion of Compound A

Preparation of co-precipitated amorphous dispersions: Co-precipitated amorphous dispersions (cPAD) were generated by precipitation of an acetone solution containing at least 50 mM of Compound A and HPMCAS-L grade (ShinEtsu) which was fed into 0.001 N HCl (cooled to 0-5° C.) on a Quadro HV0 homogenizer running at 30 m/s tip speed (to achieve an amorphous material with 30 wt. % API in a homogeneous blend with the polymer). Compound A is ulonivirine. The feed rate of acetone into the aqueous stream was controlled to roughly 15 L/hr (liters/hour) by feeding with a peristaltic pump. This cPAD material was used for each of the filtration process and the thin film evaporation process, as follows.

Preparation of spray dried intermediate: Spray dried intermediate (SDI) was manufactured on a PSD-1 spray dryer (GEA Niro, Columbia, MD, USA) equipped with a SK 80-16 atomizer nozzle (Spraying Systems Co., Glendale Heights, IL, USA). Compound A and HPMCAS-L were co-dissolved in acetone (10 wt. % solids loading) and spray dried at 15 L/hr at an inlet temperature of 119° C. The obtained SDI was dried at 40° C./15% RH for at least 12 h to remove residual solvent.

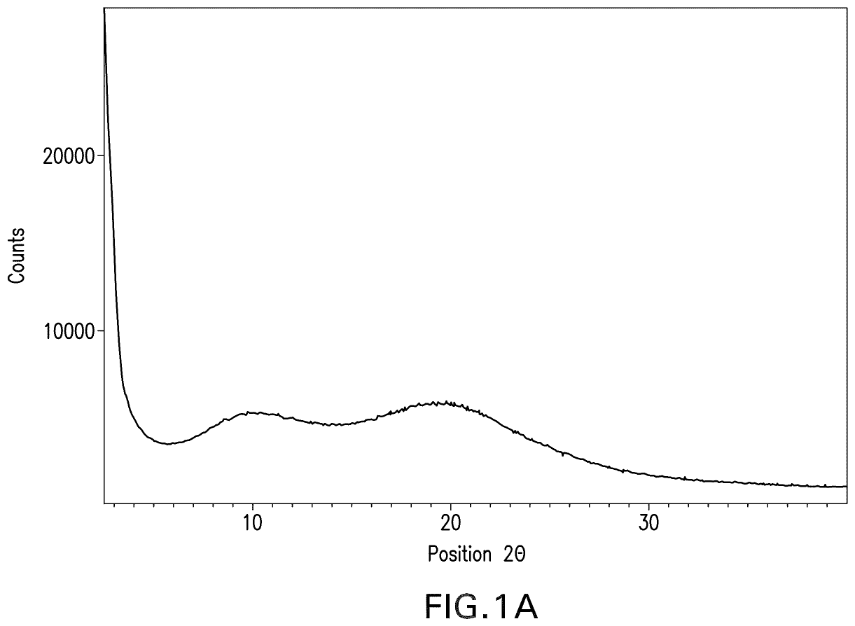

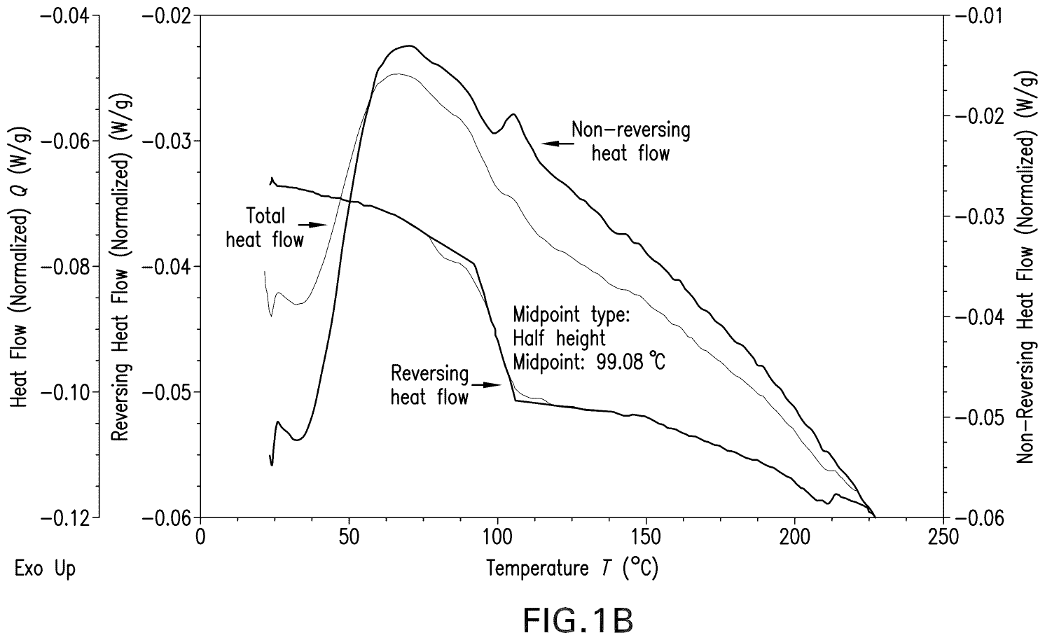

Co-precipitate isolated by filtration: The cPAD isolated by filtration and dried was shown to be amorphous by PXRD as shown in FIG. 1a and had a T_{g} at 99° C., illustrated in FIG. 1b.

Co-precipitate isolated by thin film evaporation: Preparation of the cPAD isolated by thin film evaporation (referred to herein as cPAD/TFE) was performed by feeding a slurry containing the co-precipitated material into a 0.5 square foot thin film evaporator (Artisan) using a peristaltic pump corresponding to a 2 L/hr feed rate. The thin film evaporator was brought to full vacuum (20 mmHg) at a 90-110° C. jacket temperature and the rotor was set to maximum speed for processing. The discharged cPAD from the thin film evaporator had water content between 30-60 wt. % and was dried with a nitrogen sweep to <5 wt. % solvent before downstream processing and characterization.

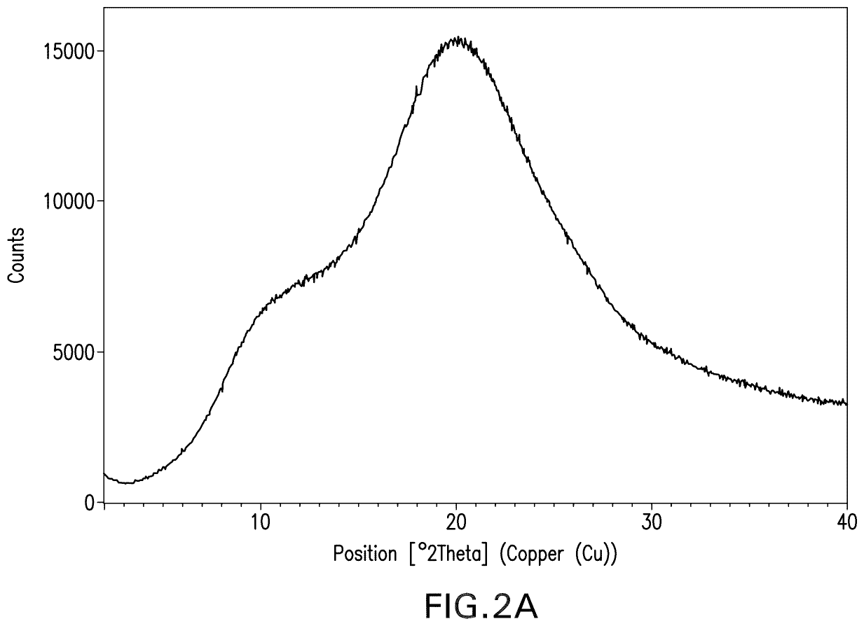

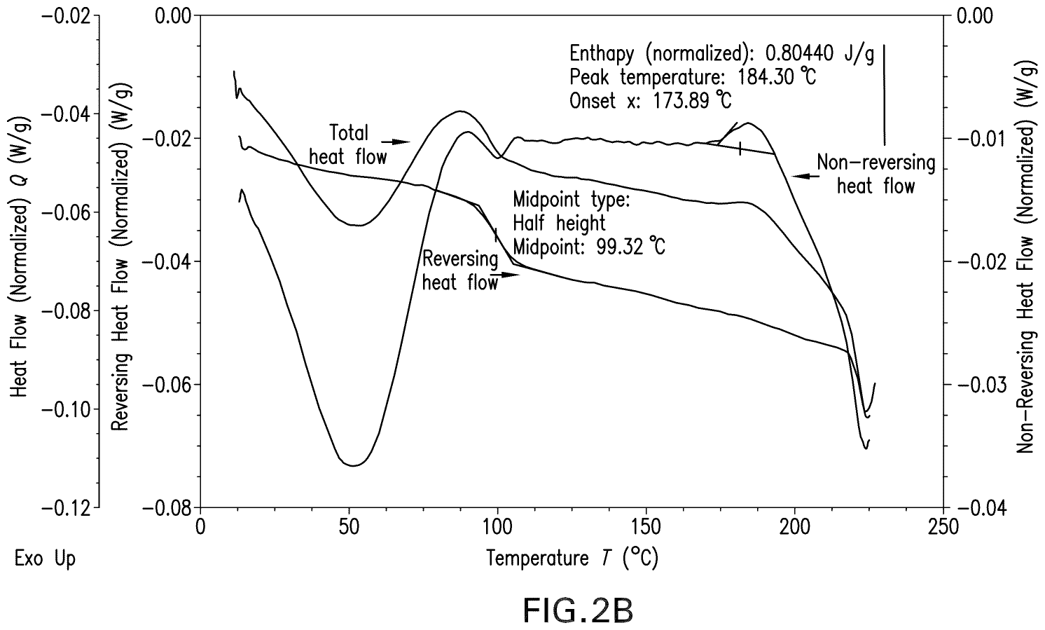

As illustrated in FIG. 2a, the TFE isolated densified cPAD/TFE was amorphous by PXRD and had a T_{g} at 99° C., illustrated in FIG. 2b, in line with the material isolated by filtration shown in FIG. 1, prior to processing by thin film evaporation.

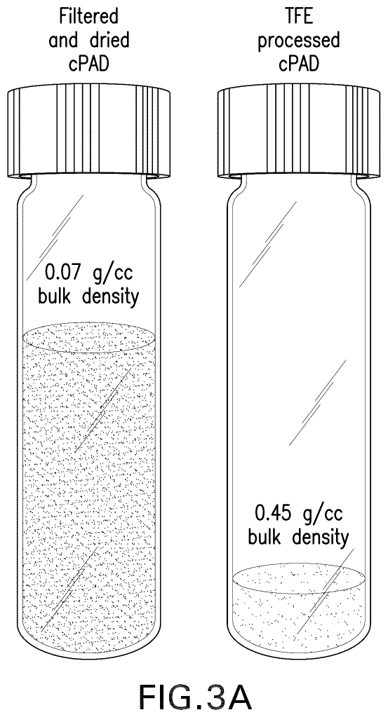

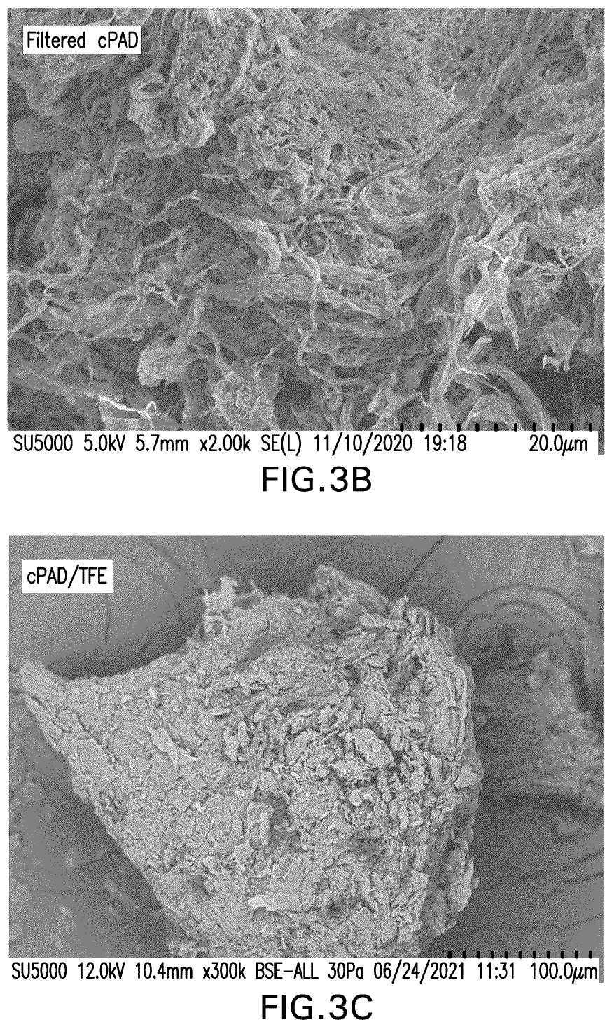

Compared to the filtered and dried cPAD, the cPAD/TFE showed improved bulk density of 0.4 g/cc (FIG. 3a). This improvement in bulk density aligns with a change in morphology. The filtered cPAD has a fibrous morphology (FIG. 3b). After thin film evaporation, the cPAD/TFE has a granular, densified morphology (FIG. 3c). Illustrated in Table 1, the precipitate isolated by thin film evaporation has improved flowability, as indicated by a smaller Flodex minimum diameter, and a larger particle size relative to the filtered and dried cPAD as well as relative to the spray dried intermediate of the same composition.

TABLE 1
Physical properties of filtered and dried cPAD compared against
cPAD isolated by thin film evaporation (cPAD/TFE).

| | Filtered and | | Spray dried | | | |
|---|---|---|---|---|---|---|
| | dried cPAD | | intermediate | | cPAD/TFE | |
| Drug loading of | 30% | | 30% | | 30% | |
| intermediate (w/w) | | | | | | |
| Bulk density (g/cc) | <0.1 | | 0.23 | | 0.45 | |
| D10 | 15.5 | μm | 9.92 | μm | 32.1 | μm |
| D50 | 67.7 | μm | 27.8 | μm | 563 | μm |
| D90 | 202 | μm | 61.0 | μm | 943 | μm |
| Flodex minimum | >34 | mm | 21 ± 2 | mm | <4 | mm |
| diameter | | | | | | |

### Example 2

Following the thin film evaporation step, the cPAD/TFE powder was formulated into tablets using direct compression, thus forming multiple compressed tablets. Table 2 compares the fit-for-purpose formulation of the Compound A spray dried intermediate described in Example 1, with the formulation for the Compound A cPAD/TFE material. The spray dried formulation required roller compaction of the SDI with lactose and Avicel® to achieve granules with acceptable material properties for tableting. With the added diluents used in this additional unit operation for the SDI, the image size for a tablet containing 60 mg pharmaceutical was 600 mg (corresponding to 10% DL Compound A in the tablet overall).

In contrast, the cPAD/TFE powder could be directly compressed into tablets without roller compaction or the addition of diluents. Enabled by the improved bulk density of the co-precipitate processed by thin film evaporation, 60 mg potency dosage units used in canine pharmacokinetic (PK) studies were only 225 mg.

TABLE 2
Tablet formulation of SDI (fit-for-purpose
formulation) and cPAD/TFE dosage units

| | cPAD/TFE | SDI | |
|---|---|---|---|
| Drug loading of the intermediate | 30% | 30% | |
| (w/w) | | | |
| Formulation | | | |
| Intermediates (w/w) | 89% | 33.33% | |
| Disintegrants (w/w) | 10.75% | 5% | |
| Diluents (w/w) | NA | Diluent 1 | 30.33% |
| | | Diluent 2 | 30.33% |
| Glidant (w/w) | NA | 0.5% | |
| Lubricants (w/w) | 0.25% | Intra- | 0.25% |
| | | granulation | |
| | | extra- | 0.25% |
| | | granulation | |
| Tablet information | | | |
| Drug loading per tablet (w/w) | 26.7% | 10% | |
| Tablet size for dog study | 225 mg | 600 mg | |
| (60 mg dose requirement) | | | |
| Tablet size for human | 750 mg | 2000 mg | |
| (200 mg dose requirement) | | | |
| | cPAD/TFE | SDI | |
| Drug loading of the intermediate | 30% | 30% | |
| (w/w) | | | |
| Formulation | | | |
| Intermediates (w/w) | 89% | 33.33% | |
| Disintegrants (w/w) | 10.75% | 5% | |
| Diluents (w/w) | NA | Diluent 1 | 30.33% |
| | | Diluent 2 | 30.33% |
| Glidant (w/w) | NA | 0.5% | |
| Lubricants (w/w) | 0.25% | Intra- | 0.25% |
| | | granulation | |
| | | extra- | 0.25% |
| | | granulation | |
| Tablet information | | | |
| Drug loading per tablet (w/w) | 26.7% | 10% | |
| Tablet size for dog study | 225 mg | 600 mg | |
| (60 mg dose requirement) | | | |
| Tablet size for human | 750 mg | 2000 mg | |
| (200 mg dose requirement) | | | |

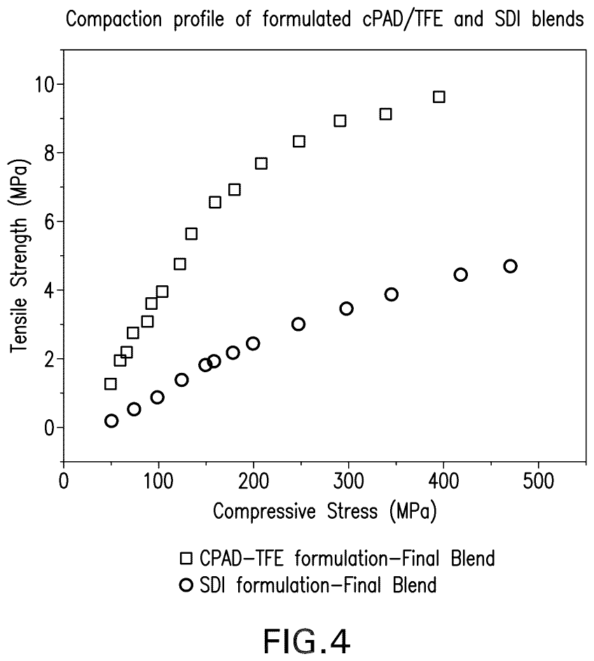

FIG. 4 compares tabletability of roller compacted granules of SDI and the directly compressed cPAD powder. Despite the formulated cPAD/TFE tablet containing far more of co-precipitated amorphous dispersion than the SDI granules, the dense material generated during thin film evaporation imparted significant strength to the tablets, allowing for a simple direct compression protocol to achieve small image size dosage forms.

### Example 3

Pharmacokinetic performance of the SDI and cPAD/TFE tablets were compared using a canine model to show equivalent pharmacokinetic performance. Mean PK parameters of Compound A dog studies are summarized in Table 3 which shows data obtained after administration of Compound A cPAD/TFE or Compound A SDI tablets in fasted, pentagastrin pre-treated Beagle dogs at a dose of 60 mg (˜6 mg/kg). There was no observed decrease in exposure after dosing the cPAD/TFE tablet relative to the spray dried material tablet. The cPAD/TFE tablets and SDI tablets were found to achieve equivalent pharmacokinetic parameters in vivo. This data suggests that cPAD/TFE formulations may find use as replacement for traditional manufacturing methods such as spray drying in cases where large doses of amorphous compound coincide to give large image size tablets that may reduce patient compliance.

TABLE 3
Bioperformance of thin film evaporated cPAD compared
with spray dried dispersion delivered.

| | | | Median | | Mean |
|---|---|---|---|---|---|
| | AUC_{0-24 hr} | Cₘₐₓ | Tₘₐₓ | Mean | Cm |
| | (ng/mL*hr) | (ng/mL) | (hr) | AUC | Cₘₐₓ |
| Formulation | (SD) | (SD) | (range) | ratio | ratio |
| Spray dried | 47815 (8354) | 2627 (487) | 2 (1-4) | Ref | Ref |
| intermediate | | | | | |
| cPAD/TFE | 47689 (3936) | 2773 (426) | 3 (2-6) | 1.0 | 1.1 |

What is claimed is:

1. A process for densifying a co-precipitated material comprised of an active pharmaceutical ingredient (API) and at least one stabilizing excipient, comprising (a) introducing a solvent stream containing dissolved API and one or more stabilizing excipient(s) into anti-solvent to form a co-precipitated material, and (b) drying and annealing the co-precipitated material above its wetted glass transition temperature.

2. The process of claim 1 wherein step (b) comprises drying and annealing the co-precipitated material above its wetted glass transition temperature by thin film evaporation.

3. The process of claim 2 wherein the co-precipitated material is a co-precipitated amorphous dispersion, nanocrystalline dispersion or microcrystalline dispersion.

4. The process of claim 3 wherein the co-precipitated material is a co-precipitated amorphous dispersion, and wherein the co-precipitated amorphous dispersion undergoes a greater level of densification relative to a process that does not anneal the amorphous dispersion above its wetted glass transition temperature.

5. The process of claim 2 wherein step (a) comprises introducing a solvent stream containing dissolved API and one, two, or three stabilizing excipient(s).

6. The process of claim 5 wherein step (a) is performed in an in-line rotor stator device, prior to (b) annealing the co-precipitated material above its wetted glass transition temperature by thin film evaporation.

7. The process of claim 2 wherein the annealing step is achieved with (a) removal of solvent, (b) increased temperature, or (c) both removal of solvent and increased temperature.

8. The process of claim 5 wherein the one, two, or three stabilizing excipient(s) are each independently selected from one, two, or three polymer(s).

9. The process of claim 5 wherein the stabilizing excipient is selected from: hydroxypropyl methylcellulose acetate succinate, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone and polyvinylpyrrolidinone-polyvinylacetate copolymers.

10. The process of claim 9 wherein the stabilizing excipient is hydroxypropyl methyl cellulose acetate succinate (HPMCAS).

11. The process of claim 5 wherein the densified co-precipitated amorphous dispersion is formulated into one or more compressed tablets.

12. The process of claim 2 wherein the API is ulonivirine.

13. A pharmaceutical composition comprising a densified co-precipitated material comprised of an active pharmaceutical ingredient (API) and at least one stabilizing excipient, wherein the co-precipitated material is annealed above its wetted glass transition temperature.

14. The pharmaceutical composition of claim 13 wherein the densified co-precipitated material is annealed above its wetted glass transition temperature by thin film evaporation.

15. The pharmaceutical composition of claim 14 wherein the densified co-precipitated material is a co-precipitated amorphous dispersion, nanocrystalline dispersion or microcrystalline dispersion.

16. The pharmaceutical composition of claim 14 comprising a co-precipitated amorphous dispersion having greater bulk density relative to a corresponding pharmaceutical composition that has not been annealed above its wetted glass transition temperature by thin film evaporation.

17. The pharmaceutical composition of claim 14 further comprising one, two, or three stabilizing excipient(s).

18. The pharmaceutical composition of claim 17 comprising one, two, or three stabilizing excipient(s) independently selected from a polymer, a polysaccharide and a polysaccharide derivative.

19. The pharmaceutical composition of claim 17 wherein the stabilizing excipient is selected from: HPMCAS, hydroxypropyl methyl cellulose phthalate, cellulose acetate phthalate, cellulose acetate trimellitate, methyl cellulose acetate phthalate, hydroxypropyl cellulose acetate phthalate, cellulose acetate terephthalate, cellulose acetate isophthalate, polyvinylpyrrolidinone and polyvinylpyrrolidinone-polyvinylacetate copolymers.

20. The pharmaceutical composition of claim 19 wherein the stabilizing excipient is HPMCAS.

21. The pharmaceutical composition of claim 17 in the form of a compressed tablet.

22. The pharmaceutical composition of claim 13 wherein the API is ulonivirine.

23. A compressed tablet comprising the pharmaceutical composition of claim claim 14.
